(19) United States
(12) Patent Application Publication
(10) Pub. No.: US 2021/0308303 A1
(43) Pub. Date: Oct. 7, 2021

(54) MEDICAL IMAGING DEVICE SUCH AS A TEE PROBE FOR UV DISINFECTION AND A DESIGN METHOD

(71) Applicant: GERMITEC, IVRY-SUR- SEINE (FR)

(72) Inventors: Clément DESHAYS, PARIS (FR); Cédric NEVEU, MONTROUGE (FR); Frédéric LÉPINE, SAINT-GRATIEN (FR)

(21) Appl. No.: 17/220,000

(22) Filed: Apr. 1, 2021

(30) Foreign Application Priority Data
Apr. 7, 2020 (FR) 20 03462

(51) Int. Cl. A61L 2/10

(52) U.S. Cl. CPC A61L 2/10; A61L 2202/10; A61L 2202/24

(57) ABSTRACT

This medical imaging device (1) such as in particular a TEE probe, able to be disinfected at least at intermediate level DNI/HLD in a UV radiation disinfection system, is characterized in that it comprises at least one part (3) made of a UV-transparent material to reduce the shadow areas on the device.

The present invention relates to a medical imaging device such as, in particular, a TEE probe and a method for designing such a medical device.

Several techniques for disinfecting this type of medical imaging device are known in the state of the art.

The simplest of these techniques consists of cleaning the device using a wipe and appropriate disinfection products.

Furthermore, there are several well-defined levels of disinfection according to the SPAULDING classification: depending on its use, a device/instrument corresponds to a level of criticality, which in turn corresponds to a level of microbiological performance.

Thus, in France, an instrument can be Non-Critical (in contact with intact skin), Semi Critical (in contact with damaged skin or mucous membrane), or Critical (in contact with sterile tissue or blood).

A Non-Critical instrument must be disinfected with a product effective against bacteria only (Low Level Disinfection, LLD), a Semi-Critical instrument with a product effective against bacteria, mycobacteria, viruses, fungi (Intermediate Level Disinfection DNI), and a Critical instrument must be sterilized (Sterilization) or, if sterilization is not possible, at least disinfected with a product as effective against spores (High Level Disinfection HLD).

From an international point of view, these requirements are similar but expressed by the terms “Low Level Disinfection” LLD for Low Level Disinfection, “High Level Disinfection” HLD for High Level Disinfection (which corresponds to the French DNI), and “Sterilization” for Sterilization (there is no DNH defined outside France, it is a local specificity).

These efficiencies against families of microorganisms are in turn framed by French, European and International standards.

For example, instruments used in so-called semi-critical situations, i.e., in contact with mucous membranes or injured skin, must undergo Intermediate Level Disinfection (ILD) in France and High Level Disinfection (HLD) internationally.

These two levels are identical, despite the apparent difference in terms, in that they require total efficacy on bacteria, fungi, mycobacteria, viruses, and partial efficacy on spores.

This DNI/HLD level is the minimum targeted by the present invention.

Several techniques make it possible to achieve this level of disinfection on medical imaging devices/instruments.

A conventional technique consists of using a bath of a chemical disinfectant in which the imaging device is soaked in order to disinfect it.

Another known technique consists of implementing a disinfection system using radiation/UV radiation and in particular UV-C, allowing at least a rapid DNI/HLD disinfection of the device.

Such a DNI/HLD disinfection by UV means that the entire surface of the medical device to be disinfected receives a UV dose sufficient to achieve this disinfection and that the intended dose is delivered during the disinfection cycle.

This also means that the surface element of the device that receives the least UV dose, also called cold spot, as opposed to a hot spot that is well exposed, must still receive a sufficient UV dose to achieve the desired disinfection and that a real-time monitoring system of the related disinfection cycles is able to know the UV dose delivered to the said cold spot.

However, tests have shown that some devices are not well suited to allow such disinfection in that they have, due to their shape, very pronounced shadow zones, or cold spots, which means that their geometric configuration presents areas that are not very accessible to UV radiation emitted in a conventional disinfection chamber.

The object of the invention is therefore to solve these problems.

To this end, the invention relates to a medical imaging device such as a TEE probe, able to be disinfected to at least intermediate level DNI/HLD in a UV radiation disinfection system, characterized in that it comprises at least one part made of a UV-transparent material in order to reduce the shadow zones on the device.

According to other features of the device according to the invention taken alone or in combination:
the said part of the device is made of a material transparent to at least 30% of the UV radiation transmission;
the said part of the device reduces the shadow areas of the device by at least 15%;
the said part of the device is made of quartz;
the said part of the device is made of polyfluoroethylene;
the said part of the device comprises at least one handling element of the device;
it is able to achieve DNI/HLD disinfection in less than 10 minutes in a UV chamber system that delivers a cumulative dose of at least 60 mJ/cm2 to at least one of the other surfaces of the device during the disinfection cycle;
the said part of the device reduces the exposure differences of its different portions by several orders of magnitude.

According to another aspect, the invention also has as its object a method for designing a medical device such as, in particular, a TEE probe as described above, characterized in that it consists in characterizing the shadow zones of the device, and in designing and validating the design of the device with the aid of a computer-assisted optical simulation tool.

The invention will be better understood upon reading the description that follows, given only as an example and made with reference to the attached drawings, wherein:

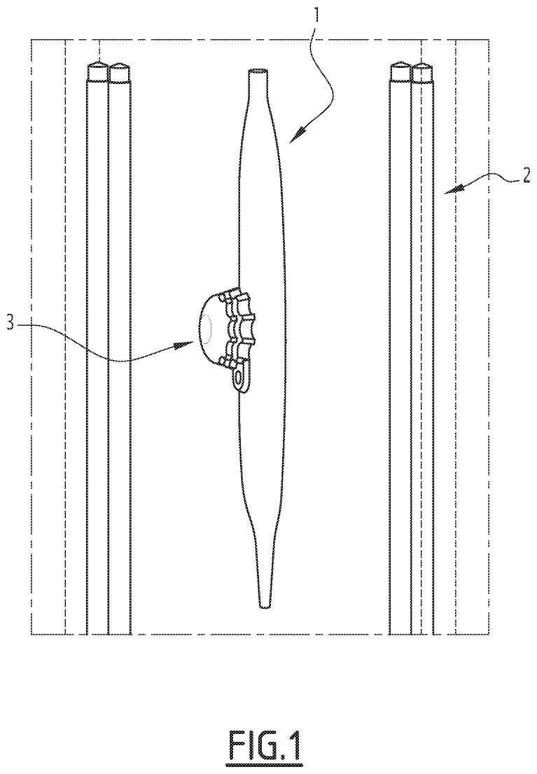

FIG. 1 shows a side view of an example of the realization of a medical imaging device in a UV disinfection system; and

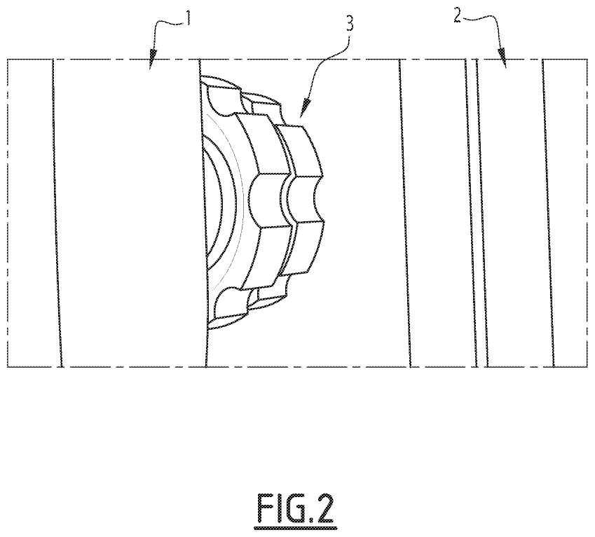

FIG. 2 shows a detailed view of this device.

Indeed, a medical imaging device has been illustrated on these figures and in particular on FIG. 1, a medical imaging device which is for example in the form of a TEE probe designated by the general reference 1 on these figures.

This device is able to be disinfected at a DNI/HLD level in a disinfection system using UV radiation, and in particular UV-C, shown in this figure and designated by the general reference 2.

As illustrated, this medical imaging device includes, for example, handling elements, designated by the general reference 3, which are likely to form shadow zones on the device, these shadow zones being also called cold spots, which do not receive a sufficient UV dose to reach the DNI/HLD disinfection.

These shadow zones of the device can for example be characterized by a computer-assisted optical simulation tool or by any other technique.

This tool can also be used to design and validate the design of such a device to verify that the designed device is able to be disinfected as required and to allow the system to monitor in real time the disinfection cycles, to calculate the dose received by and through the part transparent to the UV.

These different operations are then part of an overall method for the design and validation of the device.

In order to solve these problems of shadow zones, the medical imaging device according to the invention includes at least one part made of a UV-transparent material to reduce these shadow zones on the device.

In the example illustrated in these figures, the part or parts made of UV-transparent material of the device are handling elements of the device, i.e., for example the elements designated by the general reference 3.

These parts of the device are then made of a material transparent to at least 30% of the UV radiation transmission.

The material used can be, for example, be quartz or even polyfluoroethylene.

Of course, other materials and other parts of the device made of this type of material can also be considered.

The use of this material for at least some parts or parts of the device, which may cause shadows on the device, is intended to ensure that as much of the surface of the medical device as possible is properly exposed in order to achieve the desired DNI/HLD disinfection over the entire surface.

Thus, the said part of the device made of this UV-transparent material, allows to reduce cold spots or shadow zones by at least 15% compared to an identical configuration that would have been made of a UV-blocking material.

It allows to reduce in this sense the difference in exposure between the hot and cold spots of the device by several orders of magnitude (less than 5×, less than 4×, less than 3×, less than 2×).

This then allows the use of a conventional UV disinfection system allowing to obtain a DNI/HLD disinfection in a limited exposure time of a few minutes such as less than 10 minutes for example, in a UV chamber system that delivers a cumulative dose of at least 60 mJ/cm2 on at least one of the other surfaces of the device during the disinfection cycle.

Thus, it is conceivable that, in the device according to the invention, the cold spot will receive at least one fifth of the dose received by the hot spot during disinfection by a conventional device, the cold spot being at least twice as hot as it would have been if a material other than a UV-transparent material had been used to make the related part(s) of the device.

This can then be validated as described above by using the optical simulation tool mentioned above.

Of course, other embodiments can be considered.

1. A medical imaging device such as, in particular, a TEE probe, able to be disinfected at least at intermediate level DNI/HLD in a UV radiation disinfection system, characterized in that it comprises at least one part made of a UV-transparent material to reduce the shadow areas on the device.

2. The medical device such as, in particular, a TEE probe according to the claim 1, characterized in that the said part of the device is made of a material transparent to at least 30% of the UV radiation transmission.

3. The medical device such as, in particular, a TEE probe according to claim 1, characterized in that the said part of the device makes it possible to reduce the shadow zones of the device by at least 15%.

4. The medical device such as, in particular, a TEE probe according to claim 1, characterized in that the said part of the device is made of quartz.

5. The medical device such as, in particular, a TEE probe according to claim 1, characterized in that the said part of the device is made of polyfluoroethylene.

6. The medical device such as, in particular, a TEE probe according to claim 1, characterized in that the said part of the device comprises at least one element for handling this device.

7. The medical device such as, in particular, a TEE probe according to claim 1, characterized in that it is able to achieve DNI/HLD disinfection in less than 10 minutes in a UV chamber system which delivers a cumulative dose of at least 60 mJ/cm2 on at least one of the other surfaces of the device during the disinfection cycle.

8. The medical device such as, in particular, a TEE probe according to claim 1, characterized in that the said part of the device enables the difference in exposure of its different portions to be reduced by several orders of magnitude.

9. A method for designing a medical device such as, in particular, a TEE probe according to claim 1, characterized in that it consists in characterizing the shadow zones of the device, and in designing and validating the design thereof using a computer-assisted optical simulation tool.
